(19) United States
(12) Patent Application Publication
(10) Pub. No.: US 2026/0210969 A1
(43) Pub. Date: Jul. 23, 2026

(54) METHODS OF ANTIBODY CHARACTERIZATION

(71) Applicant: AMGEN INC., Thousand Oaks, CA (US)

(72) Inventors: Christy Ann THOMSON, Port Moody (CA); Lydia Baggott GREEN, New Westminster (CA); Wadim MATOCHKO, Burnaby (CA)

(73) Assignee: AMGEN INC., Thousand Oaks, CA (US)

(21) Appl. No.: 19/125,504

(22) PCT Filed: Oct. 30, 2023
(86) PCT No.: PCT/US2023/036224

§ 371 Date: Apr. 29, 2025

Related U.S. Application Data
(60) Provisional application No. 63/420,965, filed on Oct. 31, 2022.

(51) Int. Cl. G01N 33/68; G01N 21/552

(52) U.S. Cl. CPC G01N 33/6854; G01N 21/554

(57) ABSTRACT

The present disclosure relates to methods of characterizing low quantities of immunogen-binding protein, e.g., immunogen-binding antibodies obtained directly from supernatants of individually cultured B cells. These methods, which avoid hybridoma formation, involve providing a preparation of B cells that secrete immunogen-binding antibodies, and individually culturing the B cells under conditions suitable for the B cells to secrete the immunogen-binding antibodies into the culture supernatant. The culture supernatants containing the secreted antibodies are collected from the individually cultured B cells and subjected to one or more binding assays to characterize the immunogen-binding antibodies (e.g. as regards their binding affinity by exposing cell culture supernatants to increasing concentrations of immunogen) from the preparation of B cells.

The benefit under 35 U.S.C. § 119(e) of U.S. Provisional Application No. 63/420,965, filed Oct. 31, 2022, is hereby claimed, and the disclosure thereof is hereby incorporated by reference herein.

### FIELD

The present disclosure relates to high-throughput methods of characterizing antibody-target protein interactions using antibody containing supernatant from individually cultured B cells.

### BACKGROUND

Therapeutic monoclonal antibody (mAb) discovery typically begins with animal immunization, B cell isolation, and hybridoma formation and culture to generate large panels of mAbs per target. Identifying therapeutic candidates from these large panels involves an assessment of binding affinity, cross-reactivity, epitope binding, blocking activity, and antibody chain compositions. Given the importance of identifying candidate antibodies having the desired functional attributes, hybridoma formation and culture has been considered an essential component of the discovery process to ensure sufficient quantities of antibody are available for a thorough assessment. However, due to the lengthy time involved in hybridoma production, it can take more than 15 weeks to identify initial candidate molecules. With the high cost of developing a therapeutic mAb, the ability to identify quality leads more quickly and inexpensively would provide a significant advantage over current state of the art methods. The present disclosure is directed at overcoming these and other deficiencies in the art.

### SUMMARY

A first aspect of the present disclosure is directed to a method of characterizing immunogen-binding antibodies from a preparation of B cells. This method comprises providing a preparation of non-immortalized B cells, where B cells of the preparation secrete immunogen-binding antibodies, and culturing the B cells of the preparation individually under conditions effective for the B cells to secrete the immunogen-binding antibodies into culture supernatant. The culture supernatants containing the secreted antibodies are collected from the individually cultured B cells, and each collected supernatant is subjected to two or more different binding assays to characterize the immunogen-binding antibodies from the preparation of B cells.

Another aspect of the present disclosure is directed to a method of characterizing binding affinity of antibodies from a preparation of B cells. This method comprises providing a preparation of non-immortalized B cells, where B cells of the preparation secrete immunogen-binding antibodies. The method further involves culturing B cells of the preparation individually under conditions effective for the B cells to secrete immunogen-binding antibodies into culture supernatant. The culture supernatants containing the secreted antibodies are collected from the individually cultured B cells, and each culture supernatant is exposed to increasing concentrations of the immunogen, a fragment of the immunogen, or a homolog of the immunogen. The method further involves detecting association and dissociation between the secreted antibodies of the culture supernatant and the immunogen, fragment thereof, or homolog thereof at each of the increasing concentrations. Binding affinity of the secreted antibodies is characterized based on said detecting.

Classical methods for identifying antigen-specific antibodies involve harvesting the spleen and/or lymph nodes from immunized animals (e.g., mice), collecting B cells from the harvested tissue, and, due to challenges associated with ex vivo culture and survival of B cells, the collected B cells are immortalized. Immortalization is typically achieved via fusing the B cells with immortalized myeloma cells to produce a hybridoma. The immortalized cells are plated into wells and cultured to produce a supernatant rich in antibody that can be used in various assays such as antigen binding, affinity, blocking activity (e.g., receptor-ligand blocking), light chain determination, epitope binning, etc. to select lead antibodies possessing the desired characteristics (see FIG. 1A). Following selection of leads, sequencing is initiated to resolve the sequence of the antibody heavy and light chains required for binding and sequence transfer for recombinant production.

The primary disadvantage of the hybridoma technology is time. The protocol, excluding immunization, takes ~4-6 months to identify lead candidate antibodies (see Pedrioli, A. and Oxenium, A., “Single B Cell Technologies for Monoclonal Antibody Discovery,” Trends Immunol. 42(12): 1143-1158 (2021)). Additionally, the procedure can be low throughput in terms of fusion efficiency and hybridoma formation and some hybridomas are low-yield antibody producers.

To reduce the time involved and other disadvantages associated with hybridoma technology, advances have been made in defining culture conditions for primary B cells that avoid immortalization and allow for single B cell screening methodologies. Many of these screening approaches involve placing individual B cells in miniaturized wells or chambers, microcapillaries, or water-in-oil droplets, which contain a detectable antigen (either in suspension or immobilized) to quickly identify binding specificity of the B cell antibodies (see review article by Pedrioli, A. and Oxenium, A., “Single B Cell Technologies for Monoclonal Antibody Discovery,” Trends Immunol. 42(12): 1143-1158 (2021) at 1151-1153 and FIG. 2). Once B cells of interest are identified, the cells are retrieved and the antibody-coding sequences are obtained for recombinant production (see FIG. 1B). While these approaches are high-throughput to facilitate expeditious identification of antigen specific antibody producing cells, they have thus far been unable to accommodate functional assays, e.g., binding affinity, that are necessary to identify potential lead antibodies (see id. at 1153).

The process described herein was developed and implemented to overcome the above noted deficiencies in the antibody discovery processes. In this process, non-immortalized B cells, e.g., primary B cells isolated from an immunized animal, are plated individually into wells. The B cells are cultured to allow for secretion of antibody into the culture supernatant, which is subsequently collected to screen the secreted antibodies for a multitude of desired characteristics, including binding specificity, binding affinity, epitope binding, blocking activity, light chain composition, and other protein based interactions in one experimental run (see FIG. 1C and FIG. 2). A key difference between the historical hybridoma technology and the process described herein is that the B cells in the process of the present disclosure are not immortalized prior to antibody characterization, thereby significantly reducing the time and resources involved in the process. The process of the present disclosure also differs from current single B cell screening strategies by facilitating assessment of two or more functional characteristics, including binding affinity, which allows for candidate lead selection at a significantly earlier stage in the discovery process, i.e., prior to sequencing and cell-based recombinant production of the antibodies. Additionally, because B cell culture in the method described herein is separated from the screening process, no special technique for retrieving a desired antibody secreting cell from the screening assay is required.

Thus, the methods described herein provide a substantial improvement to the antibody discovery process by identifying a method for assaying several functional endpoints using a nominal amount of antibody, such as that produced by an individually cultured B cell. Because desired functional characteristics are identified without the need for hybridoma formation or antibody sequencing, the presently described method significantly reduces the time and resources required to identify candidate lead antibodies worthy of further therapeutic development.

### BRIEF DESCRIPTION OF THE FIGURES

FIGS. 1A-1C provide a schematic comparison of prior art methods of antibody discovery and lead selection (FIGS. 1A and 1B) versus the optimized antibody discovery and lead selection process disclosed herein (FIG. 1C). As shown in FIG. 1A, the standard hybridoma screening flow involves harvesting B cells from immunized mice and hybridoma cell formation. The media from cultured hybridoma cells is screened for antibodies with the desired immunogen binding, binding affinity, function, etc. This process involving hybridoma formation takes upwards of 15 weeks to complete. FIG. 1B shows an improvement in the process that involves antigen binding screening performed on antibodies produced from individual B cells. Once B cell producing antibodies of interest are identified, the antibodies are sequenced and recombinantly produced to provide sufficient quantities of antibodies to carry out the additional characterization of binding affinity, function, etc. The process of FIG. 1B typically takes about 7 weeks to complete. In contrast, the process of the present disclosure as depicted in FIG. 1C, involves obtaining a preparation of B cells, e.g., by harvesting B cells from immunized mice, and directly culturing the collected B cells without hy bridoma production or recombinant production. Despite the supernatant from the cultured B cells containing a limited concentration of secreted antibodies (due to the terminal nature of primary B cells), the method described herein allows for assessing two or more binding characteristics, including binding affinity, of the secreted antibodies in the supernatant. Candidate lead antibodies are identified using this process in only 3 weeks.

FIG. 2 provides a schematic depiction of how surface plasmon resonance (SPR) data obtained from sequentially performed characterization assays can be utilized to identify lead immunogen-binding antibodies obtained directly from primary B cells in a single experiment (approximately 11 hours). In this example, a lead antibody is one that does not exhibit cross-reactive binding to an off-target protein, exhibits high affinity binding to the target protein, blocks receptor binding to the target protein, and possess kappa light chains.

FIG. 3 is a graph showing the load signal response units (RU) of titrating concentrations of antibody samples, i.e., 500, 100, 50, and 25 ng/ml of antibody, loaded on the HC30-M chip (Carterra® LSA) coupled with anti-human Fc antibody (mAb 1.35.1). The 500, 100, and 50 ng/ml samples were printed on the chip for the standard 10 minute period, while the 25 ng/ml sample was printed for 30 minutes to allow for capture of the small amounts of immunoglobulin present.

FIG. 4 is a series of SPR imaging (SPRi) sensorgrams showing binding affinity measurements obtained with the Carterra® LSA instrument using the titrated antibody samples (500, 100, 50, and 25 ng/ml antibody) printed on the HC30-M chip. As noted above, the 25 ng/ml sample was printed for 30 minutes. For affinity measurement, the antibody target protein was sequentially injected at six concentrations (100 nM, 33.33 nM, 11.11 nM, 3.7 nM 1.23 nM and 0.41 nM). Association was for 10 minutes, with a 20 minute dissociation time. The data was generated using the Carterra® Kinetic software tool. Each sensorgram shows response units (RU; y-axis) over time in second (x-axis).

FIG. 5 is a collection of sensorgram reads demonstrating the output of an experiment performed according to the methods described herein and Example 2 where a panel of mAbs secreted from primary B cells were captured by a secondary antibody on a HC30-M chip. The panel of immobilized mAbs was sequentially assessed for (i) binding to an off-target protein (column 3), (ii) binding affinity to target protein (columns 7-12), (iii) blocking binding of receptor to target (column 13), and finally for light chain composition by binding to either an anti-kappa (column 15) or anti-lambda (column 16) mAb.

FIG. 6 shows utilizing the method of the present application to detect and distinguish cross-reactive binding activity of immunogen-binding antibodies collected from B cell supernatants. The SPRi sensorgrams obtained with the Carterra® LSA instrument shows binding responses of captured antibody samples upon the introduction of an off-target protein as described in Example 2 and the full experimental data shown in FIG. 5. Binding to the off-target protein by a population of mAbs is easily identified through the increased SPR signal (RU).

FIG. 7 shows representative binding affinity SPRi sensorgrams for several immunogen-binding antibodies collected from primary B cell supernatants as described in Example 2 and from the full experimental data depicted in FIG. 5. Five concentrations of target protein at a 1:3 dilution series from 100 nM were introduced to a HC30-M Carterra® chip comprising the immunogen-binding antibody supernatant samples. Association was for 10 minutes, with a 20 minute dissociation time. The data was generated using the Carterra® Kinetic software tool. Each sensorgram shows response units (RU; y-axis) over time in second (x-axis). The top panel of sensorgrams shows very high affinity antibody candidates (<100 pM) and the middle and bottom panels show high to moderate affinity antibody candidates as evidenced by the discernable off-rates.

FIG. 8 shows utilizing the method of the present application to detect and distinguish Receptor-Ligand (RL) blocking activity of representative immunogen-binding antibodies collected from primary B cell supernatants as described in Example 2 and from the full experimental data depicted in FIG. 5. The SPRi sensorgram shows the binding responses following introduction of target protein to the HC30-M chip containing the immunogen-binding antibodies (time=0 to ~2600 seconds) followed by the binding response when target receptor protein is introduced (time=~2600 to 5000 seconds). Antibodies that block receptor binding to target protein show negligible binding (i.e., negligible increase in RU) when receptor is introduced. Note that because the receptor is significantly larger than the target protein in this instance, the binding signal is likewise larger.

FIG. 9 shows utilizing the method of the present application to determine light chain composition of the immunogen-binding antibodies collected from primary B cell supernatants. The middle and right sensorgrams show binding responses for select immunogen-antibody samples following the introduction of anti-kappa antibody (middle sensorgram) or anti-lambda antibody (right sensorgram). The sensorgram on the left is the response of a buffer-only control.

FIG. 10 is a collection of sensorgram reads demonstrating the output of a second experiment performed according to the methods described herein and Example 3 where a panel of mAbs (antibodies that bind IL-11) secreted from primary B cells were captured by a secondary antibody on a HC30-M chip. The panel of immobilized mAbs was sequentially assessed for binding affinity to target protein (IL-11) (columns 2-7) and ability to block binding of receptor (IL-11R) to the IL-11 target (column 8).

FIGS. 11A-11C shows representative binding affinity SPRi sensorgrams for high affinity (FIG. 11A), medium affinity (FIG. 11B) and low affinity (FIG. 11C) IL-11 binding antibodies collected from primary B cell supernatants as described in Example 3 and from the full experimental data depicted in FIG. 10. Five concentrations of target protein at a 1:3 dilution series from 100 nM were introduced to a HC30-M Carterra® chip comprising the immunogen-binding antibody supernatant samples. Association was for 10 minutes, with a 20 minute dissociation time. The data was generated using the Carterra® Kinetic software tool.

FIG. 12 is a table summarizing the affinity data and blocking activity of three candidate IL-11 antibodies determined using the methods disclosed herein.

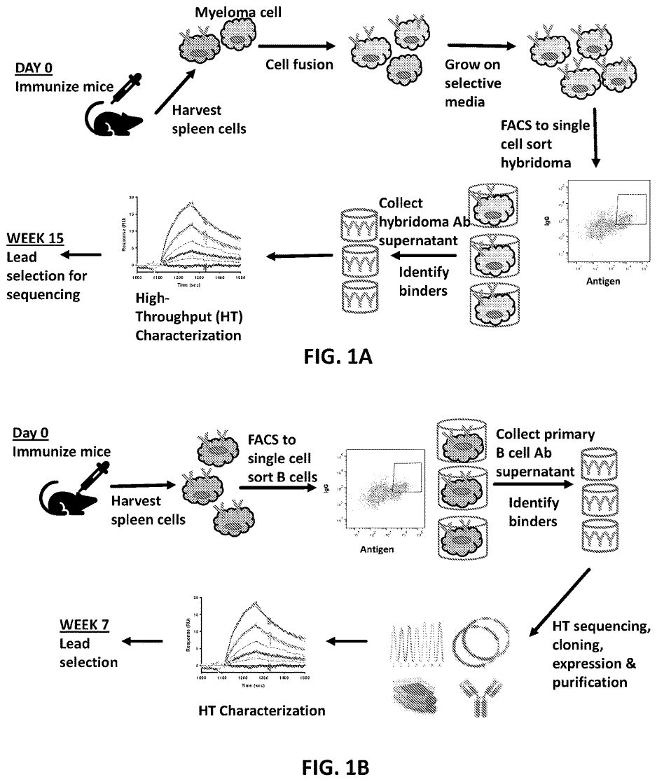

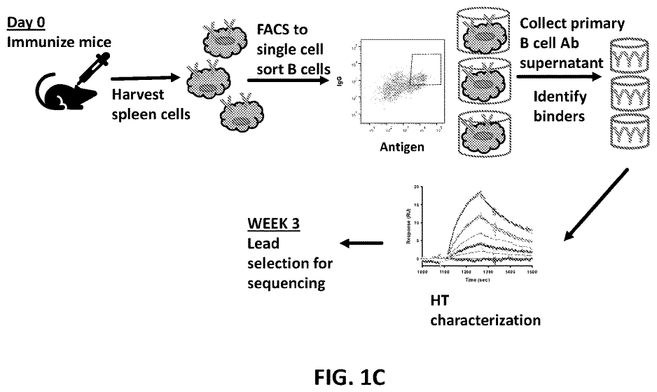

### DETAILED DESCRIPTION

The present disclosure describes the development of methods and assays that enable early biochemical characterization and lead selection of binding proteins, such as antibodies, from samples comprising a nominal amount of binding protein, such as about 1 ng to about 250 ng of total binding protein. The ability to forego methods such as hybridoma formation and cell-based recombinant production of the binding protein to generate larger quantities of binding protein prior to characterizing attributes like binding affinity, blocking activity, cross-reactivity, epitope binding, as well as other characteristics of binding proteins saves a significant amount of time and resources and provides functional information required to identify lead binding proteins suitable for advancement.

Accordingly, the present disclosure is directed to methods of characterizing binding proteins, such as antibodies, from samples comprising a nominal amount of a binding protein. In a first aspect, this method involves characterizing immunogen-binding antibodies secreted from a preparation of B cells, where the B cell supernatant comprises a nominal amount of the secreted immunogen-binding antibody. This method comprises providing a preparation of non-immortalized B cells, where B cells of the preparation secrete immunogen-binding antibodies. The method further involves culturing the B cells of the preparation individually under conditions effective for the B cells to secrete the immunogen-binding antibodies into culture supernatant. The culture supernatants containing the secreted antibodies are collected from the individually cultured B cells, and each collected supernatant is subjected to two or more different binding assays to characterize the immunogen-binding antibodies from the preparation of B cells.

In accordance with this and all aspects of the disclosure, binding assays that are suitable for functionally characterizing the immunogen-binding antibodies from the preparation of non-immortalized B cells include, without limitation, binding assays that characterize antibody binding affinity, antibody binding avidity, antibody cross-reactive binding (e.g., cross-reactive binding to the same immunogen in different species or cross-reactive binding to structurally similar immunogen), antibody blocking activity (e.g., blocking immunogen binding to its cognate binding partner), immunogen binding conditions (e.g., optimal pH conditions for antibody-immunogen binding), antibody chain composition (e.g., antibody light chain composition), antibody epitope binding, antibody-on-antibody cross-competition, and any combination of the aforementioned assays. Exemplary assays and reagents for carrying out these assays are disclosed infra.

In any embodiment, the two or more binding assays employed to characterize the immunogen-binding antibodies are carried out on a solid support, e.g., a biosensor chip. The solid support comprises a plurality of reaction surfaces, where each reaction surface comprises a capture reagent immobilized to the surface. In accordance with this embodiment, each of the collected culture supernatants is contacted with the solid support under conditions effective for secreted antibodies from a collected culture supernatant to bind to the immobilized capture reagent on a reaction surface to form an array of captured antibodies. Thus, each reaction surface on the solid support contains an immobilized antibody from a different culture supernatant. The array of captured antibodies is exposed to a first binding analyte, and the presence or absence of an interaction between the first binding analyte and the captured antibodies is detected to determine a first binding characteristic of the antibodies. The method further involves repeating the exposing and detecting steps with a second binding analyte to determine a second binding characteristic of the antibodies. The exposing and detecting steps can be repeated using third, fourth, fifth, etc. binding analytes to determine additional binding characteristics of the antibodies. Preferably, the array of captured antibodies is exposed to the binding analytes (i.e., the first, second, third, etc. binding analytes) in an order that requires minimal or no washing steps between different binding analytes and does not require replenishing the immobilized antibody on the reaction surface. This allows for multiple functional characteristics of the antibodies to be determined from one supernatant sample in one experimental run. Exemplary reaction orders that achieve these goals are described in more detail herein.

In accordance with this and all embodiments of the disclosure, solid supports suitable for immobilizing immunogen-binding antibodies from the preparation of non-immortalized B cells for characterization can be formed from any porous or non-porous material, such as silica, glass, metal, plastic, or polymers. For example, the solid support may comprise a material such as metal, glass, ceramic, silica, a polymeric material (e.g., poly(methyl methacrylate) (PMMA), polystyrene, polycarbonate, and cycloolefin copolymers (COC)), or any combination of these materials. The solid support may comprise a solid surface or a surface of optical fibers. The solid support, if not metal, may be coated in a metal, e.g., as gold, platinum, silver, or metal nanoparticles, rendering it compatible with label-free, real-time detection systems, e.g., surface plasmon resonance platforms and biolayer interferometry platforms.

The surface of the solid support, which may be coated with gold, silver, or another sensor compatible metal, may also be functionalized to facilitate or enhance immunogen-binding antibody attachment. Materials suitable for functionalizing the surface of the solid support include, without limitation polymeric materials such as poly carboxylate hydrogel or carboxymethyldextran hydrogel. In any embodiment, the functionalized surface further comprises an immobilized capture reagent, e.g., streptavidin, protein A/G, one or more poly-nitrilotriacetic acid groups, a capture antibody, or other binding moiety suitable for immobilizing the immunogen-binding antibodies of the culture supernatant to the solid support surface.

Solid supports suitable for immobilizing antibody from the culture supernatants or other samples as described herein are commercially available, see e.g., the polycarboxylate and carboxymethyldextran hydrogel sensor chips available from Carterra® Inc.

In any embodiment, the solid support surface comprises an immobilized capture reagent, e.g., streptavidin, protein A/G, one or more poly-nitrilotriacetic acid groups, a capture antibody, suitable for immobilizing the immunogen-binding antibodies to the solid support surface. In any embodiment, the immobilized capture reagent is an antibody. In any embodiment, the immobilized capture reagent is a polyclonal antibody reagent. In any embodiment, the immobilized capture reagent is monoclonal antibody reagent. In any embodiment, the immobilized capture reagent is an antibody that binds to a constant portion of an antibody heavy or light chain, e.g., the Fc portion of an antibody heavy chain. In any embodiment, the immobilized capture reagent is an anti-Fc specific antibody selected from an anti-IgG antibody, an anti-IgM antibody, an anti-IgD antibody, an anti-IgE antibody, or an anti-IgA antibody. In any embodiment, the antibody is an anti-human Fc specific antibody, e.g., an anti-human IgG antibody, an anti-human IgM antibody, an anti-human IgD antibody, an anti-human IgE antibody, or an anti-human IgA antibody. Anti-Fc antibodies that are suitable for use in the methods of the present disclosure are readily known in the art and commercially available (see e.g., anti-Fc antibodies available from, for example and without limitation, R&D Systems and SouthernBiotech). Selection of a suitable anti-Fc specific antibody will vary depending on the heavy chain composition of the immunogen-binding antibodies. Identifying a suitable capture antibody can be carried out using methods known in the art, where suitable antibodies encompass those having a high binding affinity, e.g., <200 pM, <150 pM, <100 pM, or <50 pMm.

In any embodiment, the immobilized antibody capture reagent is an anti-light chain specific antibody, such as an anti-kappa chain antibody or an anti-lambda chain antibody. In any embodiment, the anti-light chain antibody is specific to a human antibody light chain, e.g., an anti-human kappa light chain or an anti-human lambda light chain. Anti-light chain antibodies that are suitable for use as an immobilized capture reagent in the methods disclosed herein are readily known in the art and commercially available (see e.g., anti-lambda and anti-kappa antibodies available from, for example and without limitation, Abcam and R&D Systems). Selection of a suitable anti-light chain specific antibody will vary depending on the light chain composition of the immunogen-binding antibodies. Identifying a suitable anti-light chain capture antibody can be carried out using methods known in the art, where suitable antibodies encompass those having a high binding affinity, e.g., <200 pM, <150 pM, <100 pM, or <50 pM.

As described in more detail herein, the culture supernatants containing the immunogen-binding antibodies for analysis on the solid support are collected from individually cultured B cells. In any embodiment, the B cells are primary B cells. “Primary cells” as referred to herein are terminal cells isolated directly from an in vitro or an in vivo biological sample, e.g., tissue (e.g., spleen, lymph node), blood, plasma, serum, or bone. Importantly, the primary cells are terminal, non-immortalized cells. Accordingly, the amount of immunogen-binding antibody produced by primary B cells and secreted into the culture supernatant is limited in concentration as compared to the amount of antibody available from more typically utilized hy bridoma B cells or cell-based recombinant systems. Thus, the utilization of a suitable antibody capture reagent, e.g., an anti-Fc specific antibody, aids the capture of a sufficient amount of immunogen-binding antibody onto the solid support surface to detect the presence or absence of an interaction between the immobilized immunogen-binding antibodies and one or more binding analytes as described herein.

In accordance with this and all aspects of the disclosure, suitable B cells are antibody secreting B cells, and include any type of B cell that produces and secretes antibody. Thus, the preparation of B cells may comprise plasmablasts (short-lived plasma cells), plasma cells (e.g., long-lived plasma cells), and germinal cell (GC) B cells. In any embodiment, the preparation of B cells is a preparation of primary B cells that produce and secrete human antibodies. B cells that produce and secrete human antibodies include human B cells as well as non-human B cells that have been modified to produce human antibodies. In one embodiment, the B cells are derived from a transgenic animal, such as a transgenic mouse, that produces human B cells. Suitable transgenic mice whose B cells produce human antibodies include, without limitation, the XenoMouse®, HuMab Mouse®, VelocImmune® mice (VelociMouse®), Harbor Mice®, OmniMouse®, Alloy mouse, and Trianni mouse. Other transgenic animals capable of producing human antibodies from their B cells include, without limitation, transgenic chicken (e.g., OmniChicken®), transgenic rats (e.g., OmniRat®), transgenic llamas, transgenic rabbits, and transgenic cows (e.g., Transchromosomic (Tc) bovines) (see e.g., Brüggemann et al., Human Antibody Production in Transgenic Animals,” Arch. Immunol. Ther. Exp. 63:101-108 (2015), which is hereby incorporated by reference in its entirety). All forms of antibodies produced and secreted by B cells derived from transgenic animal models, including full immunoglobulin molecules and smaller domains thereof (e.g., VhH), are suitable for characterization in accordance with the methods described herein.

In another embodiment, the B cells produce and secrete non-human antibodies, e.g., primary B cells derived from a non-human animal that produce non-human antibodies. Suitable non-human B cells preparations include any mammalian B cells preparation. Exemplary non-human mammal B cells can be obtained from, for example, and without limitation, non-human primates, horses, pigs, cows, goats, sheep, llamas, camels, rabbits, dogs, cats, rats, guinea pigs, gerbils, and mice. In another embodiment, the B cells utilized in the methods described herein are derived from a non-human animal such as a bird (e.g., chickens and ducks), a shark, a fish, or a lamprey.

In any embodiment, the preparation of B cells is isolated or obtained from a subject immunized with the immunogen of interest. The immunized subject can be any immunized animal, for example, an immunized mammal. Suitable mammals for immunization include, without limitation, a human, non-human primate, horse, pig, cow, goat, sheep, llama, camel, rabbit, dog, cat, rat, guinea pig, gerbil, and mouse. In other embodiments, the immunized subject is a not a mammal. Suitable non-mammalian animals for immunization include, without limitation, birds (e.g., chickens and ducks), sharks, fish, or lamprey.

In any embodiment, the non-human animal may be a natural animal or a transgenic animal, e.g., a transgenic non-human animal capable of producing human antibodies. Suitable techniques for immunizing a non-human animal are known in the art. See, e.g., Coding, Monoclonal Antibodies: Principles and Practice, 3rd ed., Academic Press Limited, San Diego, CA, 1996 (which is hereby incorporated by reference in its entirety). The gene gun method described in, e.g., Barry et al., Biotechniques 16(4):616-8, 620 (1994); Tang et al., Nature 356(6365): 152-4 (1992); Bergmann-Leitner and Leitner, Methods Mol Biol 1325:289-302 (2015); Aravindaram and Yang, Methods Mol Biol 542:167-178 (2009); Johnston and Tang, Methods Cell Biol 43 PtA: 353-365 (1994); and Dileo et al., Human Gene Ther 14(1): 79-87 (2003) (which are hereby incorporated by reference in their entirety), also may be used for immunizing the non-human animal. The non-human animal may alternatively be immunized by administering cells expressing the antigen to the non-human animal or administering antigen-loaded dendritic cells, tumor cell vaccines, or immune cell based vaccines. See, e.g., Sabado et al., Cell Res 27(1): 74-95 (2017), Bot et al., “Cancer Vaccines” in Plotkin's Vaccines. 7th ed., Editors: Plotkin et al., Elsevier Inc., 2018, and Lee and Dy, “The Current Status of Immunotherapy in Thoracic Malignancies” in Immune Checkpoint Inhibitors in Cancer. Editors: Ito and Emstoff, Elsevier Inc., 2019 (which are hereby incorporated by reference in their entirety). In various instances, the immunizing may be carried out by microneedle delivery (see, e.g., Song et al., Clin Vaccine Immunol 17(9): 1381-1389 (2010) (which is hereby incorporated by reference in its entirety); with virus-like particles (VLPs) (see, e.g., Temchura et al., Viruses 6(8): 3334-3347 (2014) (which is hereby incorporated by reference in its entirety); or by any means known in the art. See, e.g., Shakya et al., Vaccine 33(33): 4060-4064 (2015) and Cai et al., Vaccine 31(9): 1353-1356 (2013) (which are hereby incorporated by reference in their entirety). Additional strategies for immunization and immunogen preparation, including, for example, adding T cell epitopes to antigens, are also suitable for use and described in Chen and Murawsky, Front Immunol 9:460 (2018) (which is hereby incorporated by reference in its entirety).

Accordingly, in any embodiment, the methods described herein may further comprise immunizing a subject with the immunogen of interest and collecting and isolating primary B cells from the spleen, lymph node, blood, and/or plasma of the immunized subject using B cell isolation techniques known in the art (see e.g., Moore et al. “Isolation of B-cells using Miltenyi MACS Bead Isolation Kits,” PLoS One 14(3): e0213832 (2019), which is hereby incorporated by reference in its entirety). The isolated B cells are then cultured individually and supernatants comprising secreted antibodies are subject to analysis as described herein.

In another embodiment, the preparation of B cells is isolated or obtained from subject having an autoimmune condition, where the B cells of the subject produce antibodies that bind a self-immunogen. Suitable subjects include mammalian and non-mammalian subjects. Exemplary mammalian subjects include, without limitation, humans, non-human primates, horses, pigs, cows, goats, sheep, llamas, camels, rabbits, dogs, cats, rats, guinea pigs, gerbils, and mice.

In another embodiment, the preparation of B cells is isolated or obtained from subject having or previously having a viral or bacterial infection, where the B cells of the subject produce antibodies that bind a viral or bacterial immunogen. Suitable subjects include mammalian and non-mammalian subjects. Exemplary mammalian subjects include, without limitation, humans, non-human primates, horses, pigs, cows, goats, sheep, llamas, camels, rabbits, dogs, cats, rats, guinea pigs, gerbils, and mice.

In any embodiment, the preparation of B cells is isolated or obtained from an in vitro immune organoid model, where the B cells of the model are exposed to immunogen for the purpose of antibody generation. The in vitro immune organoid model may comprise immune cells from any mammalian and/or non-mammalian animal, including, without limitation, a human, non-human primate, horse, pig, cow, goat, sheep, llama, camel, rabbit, dog, cat, rat, guinea pig, gerbil, mouse, bird, shark, fish, lamprey, or any combination thereof.

Once isolated or obtained, the primary B cells that produce and secrete immunogen-binding antibodies are individually cultured, i.e., a single primary B cell is placed in a cell culture well to seed a culture. The primary B cell is cultured and expanded using standard cell culturing methods appropriate for B cell culture, see e.g., Weitkamp et al., “Generation of Recombinant Human Monoclonal Antibodies to Rotavirus from Single Antigen-specific B Cells Selected with Fluorescent Virus-like Particles,” J. Immunol. Meth. 275:223-37 (2003) and Lagerkvist et al., “Single, Antigen-Specific B cells Used to Generate Fab Fragments using C40-mediated Amplification of Direct PCR Cloning,” BioTechniques 18:862-69 (1995), which are hereby incorporated by reference in their entirety.

In any embodiment, the primary B cell culture is maintained at least until the concentration of antibody in the culture supernatant is about 20 ng/mL to about 100 ng/mL or greater. A sample of the supernatant is then contacted with the reaction surface of the solid support. In any embodiment, about 50 μL to about 250 μL of the supernatant sample is contacted with the solid support, where the supernatant sample comprises, or is diluted to comprise, an antibody concentration of about 20 ng/mL to about 100 ng/mL. Accordingly, the total amount of immunogen-binding antibody that is contacted with the reaction surface of the solid support is at least about 1 ng to about 25 ng, more preferably at least about 3 ng to about 20 ng. In any embodiment, the total amount of immunogen-binding antibody that is contacted with the reaction surface of the solid support for analysis is at least about 1 ng, 2 ng, 3 ng, 4 ng, 5 ng, 6 ng, 7 ng, 8 ng, 9 ng, 10 ng, 11 ng, 12 ng, 13 ng, 14 ng, 15 ng, 16 ng, 17 ng, 18 ng, 19 ng, 20 ng, 21 ng, 22 ng, 23 ng, 24 ng, or 25 ng.

In any embodiment, about 150 μL to about 250 μL of the supernatant sample is contacted with the solid support, where the supernatant sample comprises, or is diluted to comprise, an antibody concentration of about 20 ng/mL to about 100 ng/mL. In any embodiment, the supernatant sample contacted with the solid support comprises an antibody concentration of about or at least 20 ng/mL, about or at least 25 ng/mL, about or at least 30 ng/mL, about or at least 35 ng/mL, about or at least 40 ng/mL, about or at least 45 ng/mL, about or at least 50 ng/mL, about or at least 55 ng/mL, about or at least 60 ng/mL, about or at least 65 ng/mL, about or at least 70 ng/mL, about or at least 75 ng/mL, about or at least 80 ng/mL, about or at least 85 ng/mL, about or at least 90 ng/mL, about or at least 95 ng/mL, or about 100 ng/mL.

In any embodiment, a sample of the collected culture supernatant is contacted with the solid support under conditions effective to capture a sufficient amount of secreted antibody onto the solid support surface. In any embodiment, this contacting may involve flowing the supernatant sample over a reaction surface of the solid support in a repeated manner, e.g., using continuous flow microspotting technology. Using this approach, the supernatant sample is cyclically flowed over the reaction surface under conditions suitable for the antibodies in the supernatant to bind to the capture reagent, e.g., anti-Fc antibodies, immobilized on the solid support surface. In any embodiment, the flow of the antibody containing culture supernatant is carried out in a bidirectional manner to maximize exposure of the antibodies in the supernatant to the solid support surface. In any embodiment, the flow of the supernatant over the surface is continued for a duration of at least 10 minutes. In any embodiment, the flow is continued for a duration of about 10 to about 40 minutes, e.g., for at least or about 15 minutes, for at least or about 20 minutes, at least or about 25 minutes, at least or about 30 minutes, at least or about 35 minutes, or at least or about 40 minutes. In any embodiment, the cyclical flow is continued for a duration of about 40 minutes. In any embodiment, the cyclical flow is continued for a duration of greater than 30 minutes. In any embodiment, the cyclical flow is continued for a duration of 30 to 40 minutes.

Spotting devices and methods suitable for immobilizing the secreted antibodies on a solid support surface are known in the art, see e.g., U.S. Pat. No. 10,300,450 to Gale et al., U.S. Pat. No. 8,210,119 to Gale et al., and U.S. Pat. No. 9,682,372 to Gale, which are hereby incorporated by reference in their entirety.

In accordance with the methods disclosed herein, the solid support is suitable for high-throughput analysis and thus comprises a plurality of reaction surfaces suitable for antibody immobilization. Accordingly, in any embodiment, the method allows for analysis of 96 to 384 supernatant samples. In any embodiment, the solid support comprises 96 reaction surfaces, each suitable for capture of antibodies from a different B cell culture supernatant. In any embodiment, the solid support comprises >96 reaction surfaces, each surface suitable for capture of antibodies from a different B cell culture supernatant. In any embodiment, the solid support comprises 192 reaction surfaces, each suitable for capture of antibodies from a different B cell culture supernatant. In any embodiment, the solid support comprises 288 reaction surfaces, each suitable for capture of antibodies from a different B cell culture supernatant. In any embodiment, the solid support comprises 384 reaction surfaces, each suitable for capture of antibodies from a different B cell culture supernatant.

Immobilization of antibodies from B cell culture supernatant onto the solid support forms an array of captured antibodies suitable for analyzing and detecting the presence or absence of an interaction between a first binding analyte and the captured antibodies to determine a first binding characteristic of the captured antibodies. The first binding analyte can be any analyte suitable for use in a binding assay to determine one or more characteristics of the antibodies in the culture supernatant. Suitable binding analytes include biomolecules, i.e., any molecule that is produced by a living organism, including, but not limited to, proteins, peptides, nucleic acid molecules (e.g., deoxyribonucleic acid (DNA) molecules, ribonucleic acid (RNA) molecules, hybrid DNA-RNA molecules), lipids, and carbohydrates (e.g., a mono-, di-, or polysaccharide). In accordance with the methods described herein, suitable binding analytes include (i) biomolecules made in a living organism, e.g., a cell, and isolated for use, and (ii) biomolecules made recombinantly or synthetically.

In any embodiment, the first binding analyte is a biomolecule suitable for characterizing binding cross-reactivity of the captured antibodies. The term “cross-reactivity,” as used herein, refers to the binding of an antibody to an immunogen other than the immunogen against which the antibody was raised. Cross-reactivity encompasses antibody binding to a homologous immunogen from a different species. For example, an antibody raised against a human immunogen may exhibit cross-reactive binding to the corresponding immunogen from a different species (e.g., mouse or monkey). Cross-reactivity also encompasses antibody binding to a protein (or other biomolecule) that is structurally similar (e.g., primary, secondary, tertiary, or quaternary structure) to the immunogen against which the antibody raised. For example, an antibody raised against a human immunogen may exhibit cross-reactive binding to structurally similar proteins in the same protein superfamily as the immunogen of interest.

Exemplary biomolecules suitable for characterizing cross-reactive binding activity of an antibody include, without limitation, proteins or peptides, that are homologous, i.e., similar in sequence or structure, to the target immunogen bound by the antibody. In any embodiment, the protein or peptide is the same immunogen, but from a different species, e.g., a human antibody that binds specifically to a human immunogen can be assessed for cross-reactive binding to the cynomolgus monkey homolog of that human immunogen. In another embodiment, the protein or peptide is a protein or peptide that is in the same or a related protein family as the immunogen and/or shares amino acid sequence or structure with the target immunogen.

In another embodiment, the binding analyte is a biomolecule suitable for characterizing binding affinity or binding avidity of the captured antibodies. The term “binding affinity” as used herein refers to the strength of the interaction between two molecules, e.g., an antibody and the epitope of its antigen. This interaction may include hydrogen bonding, ionic bonds, Van der Waals interactions and electrostatic interactions. Binding affinity is typically measured and reported in terms of the equilibrium dissociation constant (K_{D}) of the interaction. This dissociation constant is calculated by dividing the rate at which an antibody-immunogen complex dissociates (k_{off}) by the rate at which the antibody-immunogen complex forms (kₒₙ) and is expressed as a molar concentration (M). K_{D} and affinity are inversely related. Therefore, the lower the K_{D} value, the greater the binding affinity of the antibody for its antigen.

The term “binding avidity” describes the measure of overall or accumulated strength of a protein-protein complex, i.e., the total strength of all non-covalent interactions between an antibody and its antigen. Binding avidity is determined by three parameters: (i) the binding affinity of the antibody-antigen complex, (ii) the valency of the antibody, and (iii) the structural arrangement of the antibody and its antigen in the complex.

Suitable binding analytes for assessing binding affinity and avidity of the captured antibodies include the immunogen of the antibody or a fragment of the immunogen comprising the epitope bound by the antibody. When cross-reactive binding to homologous immunogen is desired, e.g., cross-reactive binding to the cynomolgus homolog of a human immunogen is often desired, binding affinity and/or binding avidity to that homologous immunogen or a fragment thereof can also be assessed.

In another embodiment, the binding analyte comprises one or more biomolecules suitable for characterizing blocking activity of the antibody. “Blocking activity” of an antibody as used herein refers to the ability of the antibody to block or disrupt the binding of the immunogen to its cognate binding partner. For example, if the immunogen is a receptor ligand, the antibody has blocking activity if it blocks or disrupts the interaction between the receptor ligand immunogen and its cognate receptor. Suitable biomolecules for testing antibody blocking activity include the immunogen or fragment thereof and the binding partner of the immunogen. To test blocking activity of the immobilized capture antibodies, the array of captured antibodies is contacted with the immunogen or fragment thereof under conditions suitable for binding between the immunogen and antibody to occur. Once antibody-immunogen complexes have formed, the immunogen binding partner is introduced. If the immunogen binding partner binds to the antibody-bound immunogen, the antibody does not have blocking activity. Conversely, if the immunogen binding partner does not bind to the antibody-bound immunogen, then the antibody possesses blocking activity.

In another embodiment, the binding analyte is one or more biomolecules suitable for characterizing immunogen binding of the captured antibodies. In one embodiment, the binding analyte comprises a combination of the immunogen with a second immunogen-binding antibody. The array of capture antibodies is first contacted with its immunogen and binding between the captured antibodies and immunogen is allowed to occur. The second immunogen-binding antibody is subsequently introduced. If the second immunogen-binding antibody is unable to bind to the immunogen (that is in complex with the captured antibodies) then the capture antibody and the second immunogen-binding antibody can be “binned” together as binding to the same region of the immunogen. This type of assay is often referred to as an antibody-on-antibody cross-competition assay.

In another embodiment, the one or more biomolecules suitable for characterizing immunogen binding of the capture antibodies comprises fragments of the immunogen. In accordance with this embodiment, the array of capture antibodies is exposed, sequentially, to a series of immunogen fragments that cover the length of the immunogen. Detecting binding of one or more immunogen fragments to the immobilized capture antibody can identify the epitope region of the immunogen bound by the captured antibodies.

In another embodiment, conditions of antibody-immunogen binding can also be assessed using the array of capture antibodies. For example, if a desired trait of the antibody is that it binds the target immunogen under selective pH conditions, the immunogen can be contacted with the array of antibodies in a sequential manner, where with each introduction, the pH of the buffer containing the immunogen is incrementally increased or decreased. Detecting immunogen binding to the captured antibodies at the varying conditions will allow for selection of antibodies having the desired binding characteristics.

In another embodiment, the binding analyte is one or more biomolecules suitable for characterizing the chain composition of the capture antibodies. For example, exemplary biomolecules for characterizing the light chain composition of the capture antibodies include an anti-kappa chain antibody and/or an anti-lambda chain antibody. Light chain antibodies, i.e., anti-kappa chain antibodies and anti-lambda chain antibodies, suitable for use in the methods described herein are known in the art and commercially available (e.g., mouse and rabbit anti-human anti-kappa and anti-lambda antibodies are available from, for example and without limitation, Abcam, Invitrogen, and R&D Systems). Other exemplary biomolecules for characterizing chain composition include, without limitation, Fc-specific antibodies, e.g., an anti-lgG antibody, an anti-IgA antibody, an anti-IgE antibody, an anti-IgM antibody, an anti-IgD antibody. Fc-specific antibodies, e.g., anti-human Fc antibodies suitable for use in the methods described herein are known in the art and commercially available.

In any embodiment of the method disclosed herein, the steps of exposing the array of captured antibodies to a first binding analyte and detecting the presence or absence of an interaction between the first binding analyte and the capture antibodies to determine a first binding characteristic of the antibodies are repeated one or more times. For example, when the first binding analyte is the immunogen or a fragment thereof and binding affinity of the captured antibodies is to be characterized, the captured antibodies are repeatedly exposed to increasing concentrations of the immunogen or fragment thereof and detection of the binding association and dissociation rates at each concentration of immunogen is utilized to calculate the K_{D} value and provide a measurement of binding affinity.

In other embodiments, the exposing and detecting steps are repeated at least two times with different binding analytes, e.g., a first binding analyte and a second binding analyte, to determine at least two different binding characteristics of the antibodies in a single assay run (i.e., same captured antibody sample). In another embodiment, the exposing and detecting steps are repeated at least three times with different binding analytes, e.g., first, second, and third binding analytes, to determine at least three different binding characteristics of the antibodies in a single assay run. In yet another embodiment, the exposing and detecting steps are repeated at least four times with four different binding analytes to determine at least four different binding characteristics of the antibodies in a single assay run. Preferably, the exposing and detecting steps are carried out in an order that minimizes or eliminates washing steps and/or antibody regeneration steps in between the introduction of different binding analytes.

In any embodiment, the exposing and detecting steps are repeated two or more times in a sequential order that allows for characterizing two or more binding characteristics of the captured antibodies in a continuous manner (i.e., in one assay run using a single captured antibody sample). Importantly, as demonstrated in the Examples herein, repeating the exposing and detecting steps with a second, and optionally, third, fourth, or more binding analytes to determine additional binding characteristics of the antibodies can be carried out on a single supernatant sample, without the need to provide additional supernatant sample. Since a nominal amount of antibody is present in the supernatant from an individually cultured primary B cell, the ability to assess multiple binding characteristics from a single antibody sample is critical to obtaining sufficient characterization data to select a subset of potential lead antibodies from an antibody campaign.

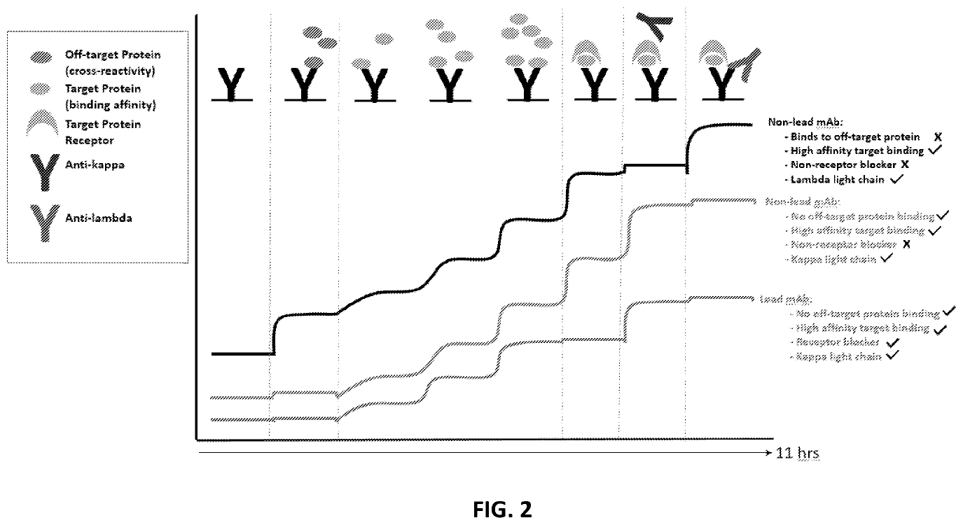

Accordingly, in one embodiment, the exposing and detecting steps are repeated in a sequential order that allows for characterizing at least cross-reactive binding activity, binding affinity, receptor-ligand blocking activity, and light chain composition (in that order). FIG. 2 provides a schematic overview of this method and the sensorgram data generated therefrom, which is further described in Example 2 herein. As shown, the combined assessment of these parameters can be utilized to identify lead candidate antibodies having the desired characteristics in less than one day (~11 hours) and from a panel of primary B cell culture supernatant samples.

In accordance with this embodiment, the first binding analyte introduced to an array of immobilized primary B cell secreted antibodies comprises one or more biomolecules suitable for characterizing binding cross-reactivity of the captured antibodies. As noted supra, exemplary first binding analytes for this purpose comprise one or more biomolecules that are homologous to the immunogen. The homologous biomolecules can be proteins, nucleic acid molecules, carbohydrates, or lipids that are structurally similar to the immunogen, e.g., a protein from the same or related protein family or protein homolog from a different species, or structurally dissimilar to the immunogen. In accordance with this embodiment, one or more different binding analytes suitable for assessing binding cross-reactivity of the captured antibodies can be contacted with the immobilized antibodies sequentially. To the extent non-desirable cross-reactive binding is detected, the immobilized antibodies bound to the cross-reactive antigen are excluded from being potential lead candidates, and thus, subsequent assays for determining binding characteristics are irrelevant for these antibodies.

The exposing and detecting steps are then repeated with increasing concentrations of a second binding analyte suitable for measuring binding affinity of the captured antibodies. As noted above, this second binding analyte can comprise the immunogen, a fragment of the immunogen, or a homolog of the immunogen.

Following exposure of the array of antibodies with the immunogen or a fragment thereof, the antibodies of interest on the array should be bound to their immunogen. To test blocking activity of the captured antibodies, the exposing and detecting steps are repeated with a third binding analyte that comprises a binding partner of the immunogen (e.g., a receptor or ligand of the immunogen). If binding of the receptor or ligand to the antibody-bound immunogen is detected, then the immobilized antibody is not a blocking antibody. If binding of the receptor or ligand to the antibody-bound immunogen is not detected, then the immobilized antibody is a blocking antibody. If binding of the receptor or ligand to the antibody-bound immunogen is detected at low levels, then the immobilized antibody may be characterized as a partial blocking antibody.

Alternatively, the third binding analyte may comprise a second immunogen-binding antibody to perform an antibody-on-antibody competition assay. As described supra, if a second immunogen-binding antibody is unable to bind the antibody-immunogen present on the array (from second analyte assay), then the immobilized antibody likely binds to the same region of the immunogen as the second immunogen-binding antibody. Accordingly, the immobilized antibody and the second immunogen-bound antibody would have the same epitope bin.

Following exposure of the array of antibodies to the third binding analyte, the immobilized antibodies may be complexed with immunogen, with immunogen and an immunogen-binding partner, or with immunogen and a second immunogen-binding antibody. Regardless of the complexed nature of the immobilized antibodies, the exposing and detecting steps can nonetheless be repeated at least an additional time with a fourth binding analyte, where the fourth binding analyte comprises one or more biomolecules suitable for characterizing light chain composition of the captured antibodies. For example, the exposing and detecting steps can be repeated with the addition of an anti-lambda chain antibody, an anti-kappa chain antibody, and a combination thereof. Binding of one of these antibodies to the immobilized antibody or antibody complex will identify the light chain identity of the immobilized antibody.

Another aspect of the present disclosure is directed to a method of characterizing binding affinity of antibodies from a preparation of B cells. This method comprising providing a preparation of non-immortalized B cells, wherein B cells of the preparation secrete immunogen-binding antibodies. The method further involves culturing B cells of the preparation individually under conditions effective for the B cells to secrete immunogen-binding antibodies into culture supernatant. The culture supernatants containing the secreted antibodies are collected from the individually cultured B cells, and each culture supernatant is exposed to increasing concentrations of the immunogen, a fragment of the immunogen, or a homolog of the immunogen. The method further involves detecting association and dissociation between the secreted antibodies of the culture supernatant and the immunogen, fragment thereof, or homolog thereof at each of the increasing concentrations, and characterizing binding affinity of the secreted antibodies based on said detecting.

In accordance with this aspect of the disclosure, this method may further comprise providing a solid support comprising a plurality of reaction surfaces, where each reaction surface comprises a capture reagent immobilized to the surface as described supra. Each of the collected culture supernatants is contacted with the solid support under conditions effective for secreted antibodies from one collected culture supernatant to bind to the immobilized capture reagent on one reaction surface to form an array of captured antibodies on the solid support. Exposing the culture supernatants to increasing concentrations of immunogen, a fragment of the immunogen, or a homolog of the immunogen and detecting the association and dissociation between the antibodies of the culture supernatants and the immunogen, fragment thereof, or homolog thereof is carried out on the solid support.

In any embodiment, this method may optionally further involve contacting the array of captured antibodies with a biomolecule that is homologous to the immunogen, and determining the presence or absence of an interaction between the homologous biomolecule and the captured antibodies to identify binding cross-reactivity of the captured antibodies. As noted above, the homologous biomolecule is any biomolecule, e.g., protein, nucleic acid molecule, lipid, or carbohydrate, that is structurally similar to the immunogen. In a preferred embodiment, cross-reactivity of the capture antibody is determined prior to determining binding affinity. Doing so can eliminate certain immunogen-binding antibodies from further analysis if the tested cross reactivity is undesirable. In another embodiment, cross-reactivity of the capture antibody is determined after binding affinity. In this approach, a wash step may be included to remove bound immunogen from the array of captured antibodies.

The method can optionally further comprise contacting the array of capture antibodies with one or more additional binding analytes after assessment of binding affinity to the immunogen, and identifying the presence or absence of an interaction between the one or more additional binding analytes and the captured antibodies to characterize one or more additional features of the captured antibodies. In any embodiment, the captured antibody is bound to immunogen (antibody-immunogen complex) at the time the one or more additional binding analytes are introduced to the solid support reaction surface.

In any embodiment, the one or more additional binding analytes is a binding partner of the immunogen, and blocking activity of the capture antibodies is characterized as described supra. In any embodiment, the one or more additional binding analytes is an agent that binds an antibody light chain and light chain composition of the captured antibodies is characterized as described supra. In any embodiment, the one or more additional binding analytes comprise another immunogen-binding antibody, and epitope binning of the captured antibodies is characterized as described supra. In any embodiment, the one or more additional binding analytes comprises a one or more immunogen fragments, and epitope mapping of the captured antibodies is characterized as described supra.

Methods for analyzing binding affinity, binding kinetics, cross-reactivity, and other binding interactions are known in the art (see, e.g., Ernst et al., Determination of Equilibrium Dissociation Constants, Therapeutic Monoclonal Antibodies (Wiley & Sons ed. 2009), which is hereby incorporated by reference in its entirety) and suitable for use in the carrying out the methods described herein. These methods include, but are not limited to, solid-phase binding assays (e.g., ELISA assay), immunoprecipitation, flow cytometry, fluorescence-activated cell sorting (FACS), surface plasmon resonance (SPR), surface plasmon resonance imaging (SPRi), e.g., Carterra® LSA (Salt Lake City, UT) and Biacore™ (GE Healthcare, Piscataway, N.J.)), kinetic exclusion assays (e.g., KinExA®), BioLayer interferometry, e.g., Octet™ (Sartorius, Fremont, CA), MicroScale Thermophoresis (MST), e.g., NanoTemper Monolith (NanoTemper Technologies GmbH, Munich Germany), and isothermal titration calorimetry (ITC), e.g., Microcal ITC200 (Malvern Panalytical, Malvern UK).

In any embodiment, detecting the presence or absence of a binding interaction of the immunogen-binding antibodies derived from primary B cells is carried out using surface plasmon resonance (SPR). SPR techniques are reviewed, e.g., in Hahnfeld et al., “Determination of Kinetic Data Using SPR Biosensors,” Methods Mol. Med. 94:299-320 (2004) and Nguyen et al., “Surface Plasmon Resonance: A Versatile Technique for Biosensor Applications,” Sensors (Basel) 15(5): 10481-510 (2015), which are hereby incorporated by reference in their entirety. In a typical SPR experiment, the B cell derived antibodies are immobilized on a SPR-active, gold-coated glass slide in a flow cell, and a sample containing one of the binding analytes described herein is introduced to flow across the surface. When polychromatic light of a given wavelength is shined on the gold surface at a particular angle (angle of incidence), a portion of the light energy excites electrons on the surface. The angle of incidence is strongly affected by the refractive index of material bound on or near the gold surface. Therefore, when a binding interaction occurs between the immobilized antibodies and a potential binding analyte, the refractive index increases and changes the angle of incidence. The change in angle of incidence is measured to produce a response curve in real-time from which the kinetics of binding can be extrapolated.

In any embodiment, binding interactions of the immunogen-binding antibodies derived from B cells is carried out using SPR imaging (SPRi) or SPR microscopy. SPRi follows the same general principles of traditional SPR, however, the information that is measured and method of detection differs slightly, allowing for a higher throughput method of studying binding interactions. In particular, a polarized light beam (as opposed to polychromatic light) is shown onto the thin gold films, and a charge-coupled device (CCD) camera is utilized to capture high-resolution images of the binding area.

As demonstrated in the Examples herein, the Carterra® LSA instrument, which employs high throughput surface plasmon resonance imaging (HT-SPRi) to measure kinetic interactions of biomolecules, is a particularly suitable platform for carrying out the methods described herein in a high-throughput manner. Other instruments that utilizes SPR detection in a high throughput manner are also suitable for detecting and measuring kinetic interactions as described herein, these instruments include, e.g., the Biacore T200 and Biacore 8K instruments (Cytiva, Marlborough, MA).

An alternatively suitable detection method that can be employed in the methods described herein involves BioLayer Interferometry (BLI) based detection. This technique is described, e.g., in Wilson et al., Biochemistry and Molecular Biology Education, 38:400-407 (2010), and Dysinger et al., J. Immunol. Methods, 379:30-41 (2012), which are hereby incorporated by reference in their entirety. BLI is an optical technique that measures macromolecular interaction by analy zing interference patterns of white light reflected from the surface of a biosensor tip. In a typical BLI experiment, the B cell derived antibodies are immobilized on the biosensor tip and the tip is introduced into a solution well containing one of the binding analytes as described herein. Binding of an analyte to the immobilized antibodies on the biosensor tip causes a shift in the interference pattern that is measured in real-time. The Octet® BLI label free detection systems from Sartorius are suitable for carrying out the methods described herein in a high-throughput manner.

As demonstrated in the Examples herein, multiple binding characteristics of immunogen-binding proteins can accurately be characterized from B cell supernatant samples comprising nominal antibody concentrations. Therefore, the methods described herein are suitable for characterizing binding proteins in other samples comprising minimal concentrations of binding proteins. Accordingly, another aspect of the present disclosure is directed to a method of characterizing binding proteins in samples comprising less than or equal to 250 ng of binding protein. In any embodiment, the method of characterizing binding proteins in accordance with the methods described herein can be carried out using samples comprising about 1 ng to about 250 ng of a binding protein, in sample comprising 1 ng to about 100 ng of a binding protein, and in sample comprising 1 ng to about 25 ng of a binding protein. This method involves contacting the samples with a solid support comprising a plurality of reaction surfaces, where each reaction surface comprises a capture reagent immobilized to said surface. Contacting the sample with the solid support is carried out under conditions effective for binding proteins in the sample to bind to the immobilized capture reagent on a reaction surface thereby forming an array of captured binding proteins on the solid support. The method further involves subjecting the array of captured binding proteins to two or more different binding assays to characterize the binding proteins in the sample.

Suitable solid supports and capture reagents for immobilizing binding proteins from a sample to form the array of captured binding proteins are described supra. As described in the Examples herein, contacting samples containing small amounts of binding protein with the solid support reaction surfaces is carried out by flowing the sample over a reaction surface of the solid support, and repeating or cycling the flow over the reaction surfaces of the solid support for at least 15 minutes. In any embodiment, the flow of the sample containing the binding protein is carried out in a bidirectional manner to maximize exposure of the binding protein in the sample to capture reagent on the solid support surface. In any embodiment, the flow of the sample over the surface is continued for a duration of at least 10 minutes. In any embodiment, the flow is continued for a duration of about 10 to about 40 minutes, e.g., for at least or about 15 minutes, for at least or about 20 minutes, at least or about 25 minutes, at least or about 30 minutes, at least or about 35 minutes, or at least or about 40 minutes. In any embodiment, the cyclical flow is continued for a duration of about 40 minutes. In any embodiment, the cyclical flow is continued for a duration of greater than 30 minutes. In any embodiment, the cyclical flow is continued for a duration of 30 to 40 minutes.

As described supra, the concentration of the binding protein in the sample is about 1 ng to about 25 ng. More preferably the concentration of the binding protein in the sample is about 3 ng to about 20 ng. To maximize immobilization of the binding protein in the sample to the solid support, a sample volume of about 50 μL to about 250 μL of a sample comprising about 20 ng/mL to about 100 ng/mL of binding protein is contacted with the reaction surface of the solid support as described above. In any embodiment, about 150 μL to about 250 μL of the sample is contacted with the solid support, where the sample comprises, or is diluted to comprise, a binding protein concentration of about 20 ng/mL to about 100 ng/mL. In any embodiment, the sample contacted with the solid support has a sample volume of about 150 μL to about 250 μL and a total amount of binding protein comprising or consisting of about 1 ng, about 2 ng, about 3 ng, about 4 ng, about 5 ng, about 6 ng, about 7 ng, about 8ng, about 9 ng, about 10 ng, about 11 ng, about 12 ng, about 13 ng, about 14ng, about 15 ng, about 16 ng, about 17 ng, about 18 ng, about 19 ng, about 20 ng, about 21 ng, about 22 ng, about 23 ng, about 24 ng, or about 25 ng.

In accordance with this aspect of the present disclosure, suitable binding proteins that can be characterized in the described method include antibodies, i.e., intact immunoglobulin molecules, immunoglobulin domains (e.g., VhH domains, unidabs) and fragments thereof (Fab, Fab′, F(ab′)2, antibody derivatives (e.g., scFv, diabody, tribody, minibody, etc.), and synthetic binders, such as minibinders.

In accordance with this aspect of the present disclosure, subjecting the array of captured binding proteins to two or more different binding assays to characterize the binding proteins in the sample is carried out as disclosed supra, i.e., the array of captured binding proteins is exposed to a first binding analyte and the presence or absence of an interaction between the first binding analyte and the captured binding proteins is detected to determine a first binding characteristic of the binding proteins. This process of exposing and detecting with second and optionally third, fourth, fifth binding analytes is repeated sequentialy to determine additional binding characteristics of the binding protein.

Importantly, as demonstrated in the Examples herein, repeating the exposing and detecting steps with a second, and optionally, third, fourth, or more binding analytes to determine additional binding characteristics of antibodies can be carried out on a single sample, without the need to provide additional sample. Preferably, the array of captured antibodies is exposed to the binding analytes (i.e., the first, second, third, etc. binding analytes) in an order as described supra that requires minimal or no washing steps between different binding analytes and does not require replenishing the binding protein on the reaction surface. This allows for multiple functional characteristics of the binding proteins to be determined from one supernatant sample in one experimental run. Exemplary reaction orders that achieve these goals are described in more detail herein.

Binding assays that are suitable for functionally characterizing the binding proteins from the sample include, without limitation, binding assays that characterize antibody binding affinity, antibody binding avidity, antibody cross-reactive binding (e.g., cross-reactive binding to the same immunogen in different species or cross-reactive binding to structurally similar immunogen), antibody blocking activity (e.g., blocking immunogen binding to its cognate binding partner), immunogen binding conditions (e.g., optimal pH conditions for antibody-immunogen binding), antibody chain composition (e.g., antibody light chain composition), antibody epitope binding, antibody-on-antibody cross-competition, and any combination of the aforementioned assays. Exemplary assays and reagents for carrying out these assays are disclosed supra.

The invention having been described, the following Examples are offered by way of illustration, and not limitation.

### EXAMPLES

### Example 1: Assay Optimization

The Carterra® LSA instrument (Salt Lake City, UT) was the instrument selected to perform binding and affinity measurements of immunogen-binding antibodies collected from the supernatant of primary B cells. Accordingly, the first step was to determine the optimal conditions to capture low concentration antibodies from the supernatants of individually cultured B cells. The three conditions optimized in this Example include immobilization of capture antibody, the minimal supernatant antibody concentration, and antibody print time.

To prepare the antibody immobilization surfaces, the Single Flow Channel (SFC) and 96-Print Head (96PH) of the Carterra® LSA instrument were primed with running buffer (Hepes buffered Steinberg's Solution (HBS-T); 50 mM HEPES pH 7.5, 150 mM NaCl, 0.1% Tween 20). The capture surface was prepared in the SFC by standard amine-coupling. An HC30-M chip (Carterra® LSA cat #4279) was activated with a 10-minute injection of freshly prepared 1:1:1 (v/v/v) mixture of 0.4 M EDC+0.1 M NHS+0.1 M MES pH 5.5. A monoclonal mouse anti-human-Fc antibody (mAb 1.35.1) was prepared at 100 μg/mL in 10 mM sodium acetate pH 4.5 (Carterra® cat #3628) and coupled to the HC30-M chip for 20 minutes. Excess reactive esters were blocked with a 7-minute injection of 1 M ethanolamine HCl pH 8.5 (Carterra® cat #3626).

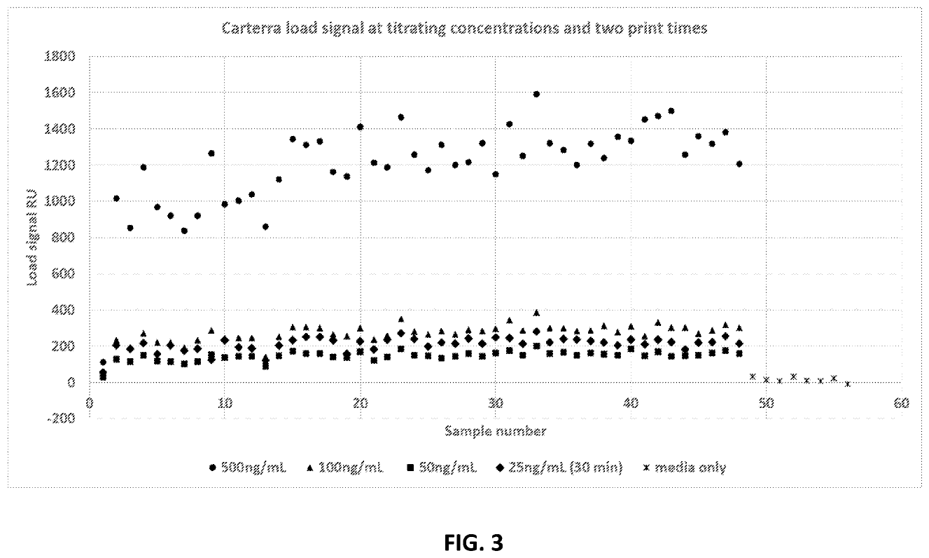

To investigate the minimal immunogen-binding antibody concentration needed and antibody print time, the Carterra® LSA 96PH was used to print immunogen-binding antibodies of known concentration for different times onto the chip coupled with mAb 1.35.1 as described above. Forty-eight antibody samples were diluted to 500, 100, 50, or 25 ng/mL in running buffer (Hepes buffered Steinberg's Solution (HBS-T); 50 mM HEPES pH 7.5, 150 mM NaCl, 0.1% Tween 20). The 500, 100, and 50 ng/mL samples were printed for 10 minutes, and the 25 ng/mL samples were printed for 30 minutes. The goal was to determine the lowest antibody concentration needed to reach at least 100 RU of antibody print signal. It was found that the 25 ng/mL antibody samples printed for 30 minutes yielded approximately 200-250 RU of signal (see FIG. 3), which was better than the RU signal obtained from printing the 50 ng/mL antibody sample on the chip for 10 minutes.

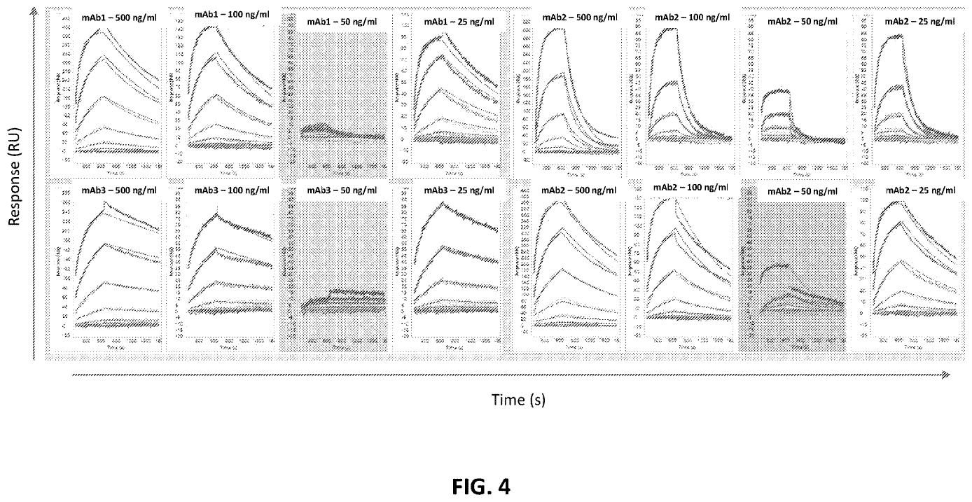

To confirm that the low level antibody concentration at longer print time was sufficient for affinity measurements, the following affinity assay was performed. After printing the antibodies at 100, 50, or 25 ng/mL as described above, a non-regenerative kinetic assay was set up by using the SFC to sequentially inject 6 concentrations of target immunogen at a 1:3 dilution series from 100 nM (i.e., 100, 33.33, 11.11, 3.7, 1.23, and 0.41 nM). Dilutions were in running buffer and injected from low to high concentration with no regeneration in between injections. Association was for 10 minutes, with a 20-minute dissociation time. The data was double referenced in that both a local reference and a zero nanomolar analyte concentration (buffer) were subtracted. The double-referenced data were fit globally to a 1:1 Langmuir binding model using the Carterra® Kinetic tool, allowing each spot its own kₐ and k_{d} value to determine K_{D}. The analysis shows that an antibody concentration of 25 ng/mL printed for 30 minutes gives similar affinity data to an antibody concentration of 100 ng/mL printed for 10 minutes (compare the 100 ng/mL and 25 ng/mL sensorgrams of FIG. 4). Data also shows that an antibody concentration of 50 ng/mL printed for 10 minutes does not capture enough antibody for good affinity measurements (see FIG. 4, shaded graphs))

### Example 2—Antibody Characterization Using Antibody-Containing Supernatant from Primary B Cells

The Carterra® LSA instrument was used to characterize cross reactivity, binding affinity, Receptor-Ligand (RL) blocking, and light chain composition of immunogen-binding antibodies collected from primary XenoMouse® B cells in one continuous experimental run (as depicted in FIG. 2). In this Example, the desired antibody characteristics included high affinity binding to the interleukin cytokine protein target (referred to herein as “target protein”) with no binding to a related interleukin cytokine (referred to herein as “off-target protein”). Additionally, it was desirable that candidate antibody binding to the target protein blocked target protein binding to its receptor.

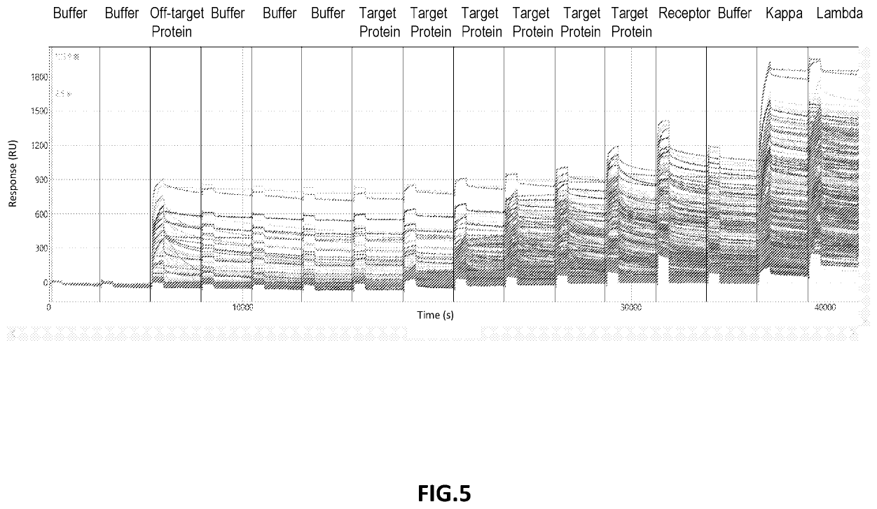

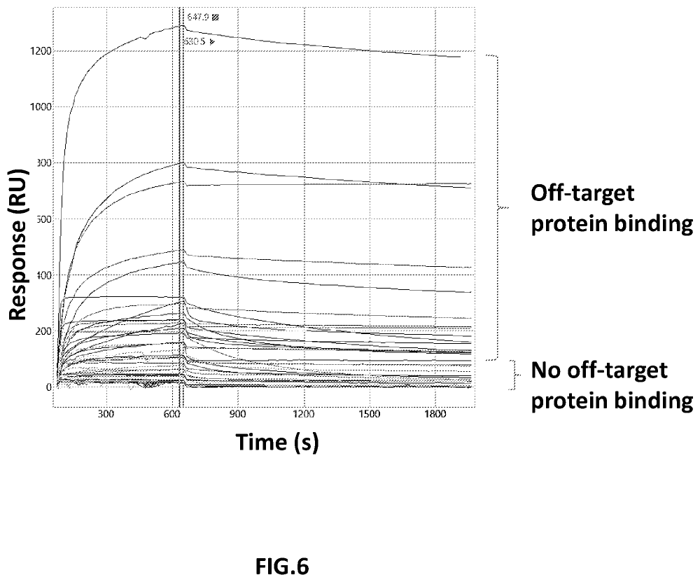

FIG. 5 shows the cumulative sensorgram of this experimental run, where each of cross-reactivity, binding affinity, RL blocking, and light chain composition of the panel of mAbs secreted from primary B cells were assessed sequentially in one run (from one B cell supernatant sample) over the course of about 11 hours. Each component of the experimental run is described in more detail below with corresponding portions of the cumulative sensorgram shown in FIGS. 6-9.

To prepare the assay surfaces, the Single Flow Channel (SFC) and 96-Print Head (96PH) of the Carterra® LSA instrument were primed with running buffer (Hepes buffered Steinberg's Solution (HBS-T); 50 mM HEPES pH 7.5, 150 mM NaCl, 0.1% Tween 20). The capture surface itself was prepared in the SFC by standard amine-coupling. An HC30-M chip (Carterra® LSA cat #4279) was activated with a 10-minute injection of freshly prepared 1:1:1 (v/v/v) mixture of 0.4 M EDC+0.1 M NHS+0.1 M MES pH 5.5. A monoclonal mouse anti-HuFc antibody (mAb 1.35.1) was prepared at 100 μg/mL in 10 mM sodium acetate pH 4.5 (Carterra, #3628) and coupled to the surface for 20 minutes. Excess reactive esters were blocked with a 7-minute injection of 1 M ethanolamine HCl pH 8.5 (Carterra, #3626). Final coupling amounts were greater than 1000 response units (RU).

Primary B cell generated antibody samples comprising >100 ng/mL of antibody were diluted ~1:4.5 (40 μL of supernatant plus 140 μL of running buffer). Diluted samples were printed on the HC30-M chip surface comprising the mAb 1.35.1 antibody for 30 minutes using the 96PH.

After printing, a 100 nM injection of the off-target protein using the SFC was used to screen for cross reactivity of the immobilized immunogen-binding antibodies. FIG. 6 shows a SPRi sensorgram (graph of Response Units (RU) versus time) for various antibody samples. Binding to the off-target protein of a population of mAbs is easily identified through the increased SPRi signal (RU) upon off-target protein introduction.

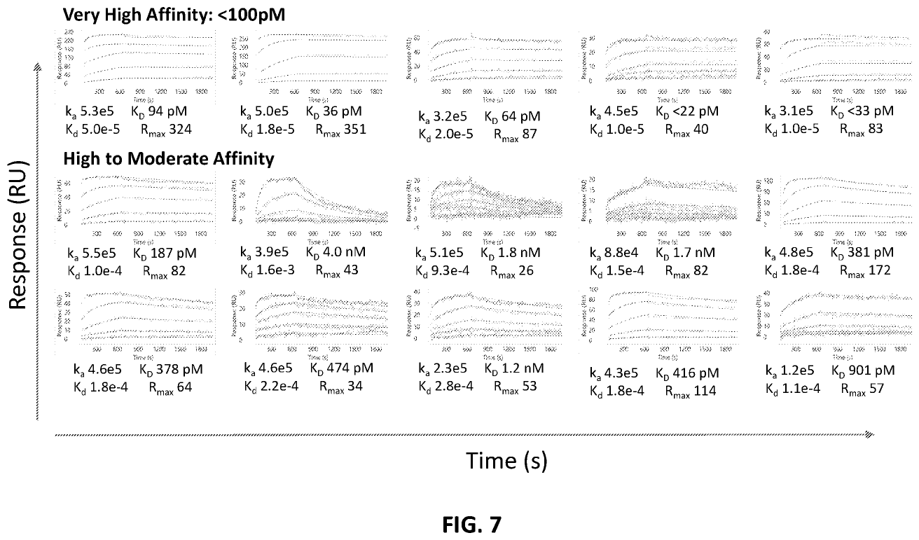

Next a non-regenerative kinetic assay was set up by using the SFC to sequentially inject 6 concentrations of target protein at a 1:3 dilution series from 100 nM (i.e., 100, 33.33, 11.11, 3.7, 1.23, and 0.41 nM). Dilutions were in running buffer and injected from low to high concentration with no regeneration in between injections. Association was for 10 minutes, with a 20-minute dissociation time. FIG. 7 shows the affinity sensorgrams for several of the tested antibodies. The top panel of sensorgrams show high affinity binders (i.e., K_{D}<100 pM) and the middle and bottom panels show high to moderate affinity antibody candidates as evidenced by the discernable off-rates.

Binding of the antibodies to the off-target protein could potentially interfere with the affinity measurements. However, because off-target binding was not desired, any antibodies exhibiting cross-reactive binding to the off-target protein were immediately eliminated from advancement. Accordingly, any affinity or R-L blocking data (described below) was considered irrelevant to antibodies exhibiting off-target protein binding.

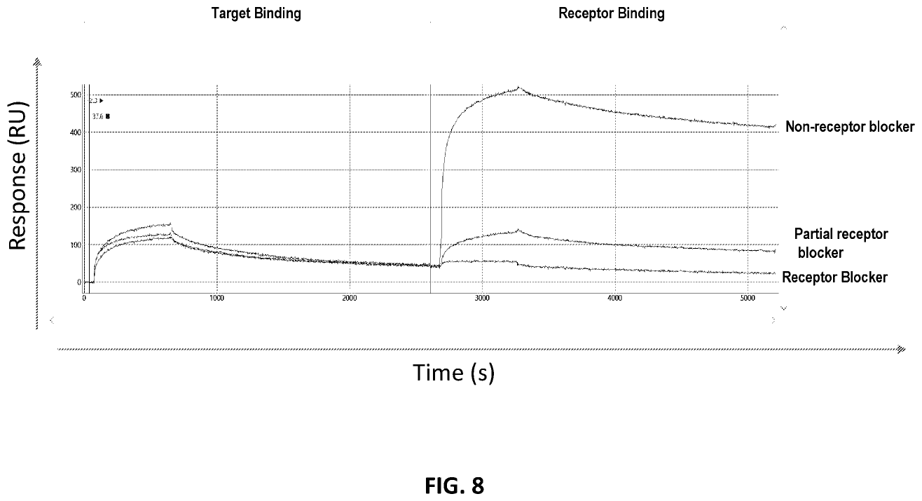

Immediately after the kinetics cycle, a single 10 minute injection of the target protein receptor at 100 nM in running buffer was performed to measure R-L blocking activity of the immunogen-binding antibodies. The sensorgrams of FIG. 8 show the response of a non-blocking antibody, a partial blocking antibody, and a full blocking antibody. The SPR sensorgram first shows the binding responses following introduction of target protein to the HC30-M chip containing the immunogen-binding antibodies (see “Target Binding” portion of sensorgram). The target protein receptor was then introduced to the chip, and the binding response recorded (see “Receptor Binding” portion of the sensorgram). Antibodies that block receptor binding to target protein show negligible binding response when the target protein receptor is introduced. Note that because the target protein receptor is significantly larger than the target protein, the binding signal generated from receptor-target binding is likewise larger.

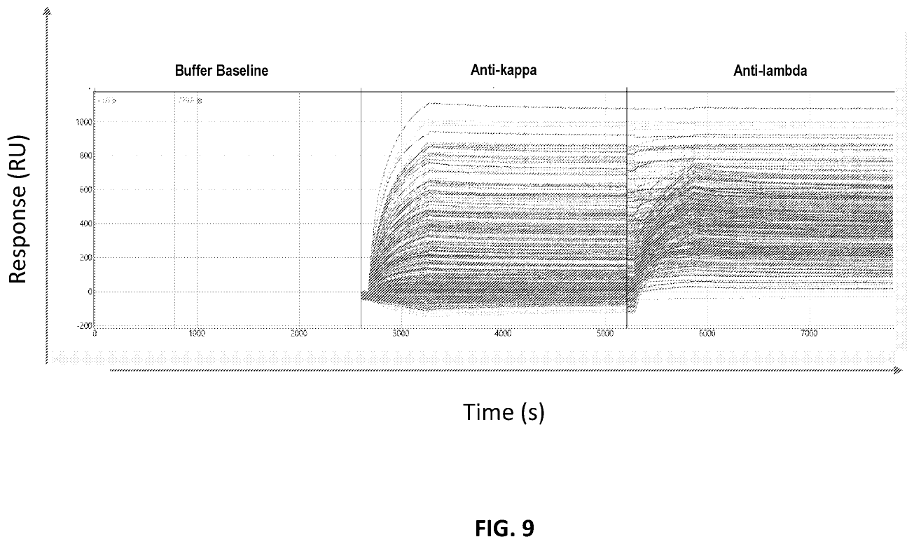

Finally, sequential injections of anti-human kappa and anti-human lambda antibodies at 5 μg/mL for 20 minutes allowed for identification of the light chain subtype of the immunogen-binding antibodies as shown in the sensorgram of FIG. 9.

Based on the data generated in this experimental run of 1,222 antibody samples, it was determined that 1021 (83.6 %) of the antibodies did not exhibit cross-reactive binding to a non-target protein, 182 of the antibodies (14.9%) exhibited the desired high affinity target protein binding (i.e., affinity of <100 pM), and 26 of these antibodies (2.1%) were target protein receptor blocking antibodies. Accordingly, of the 1,222 antibodies that were screened, this method of screening identified 26 potential lead candidates having the desired binding specificity, affinity, and blocking activity from primary B cell supernatant in just over 11 hours.

### Example 3—IL-11 Antibody Characterization Using Antibody-Containing Supernatant from Primary B Cells

In this Example, the Carterra® LSA instrument was used to characterize binding affinity and Receptor-Ligand (RL) blocking activity of immunogen-binding antibodies collected from primary XenoMouse® B cells. The target protein of the B cell secreted antibodies of this Example was interleukin 11 (IL-11).

To prepare the assay surfaces, the Single Flow Channel (SFC) and 96-Print Head (96PH) of the LSA instrument were primed with running buffer (Hepes buffered Steinberg's Solution (HBS-T); 50 mM HEPES pH 7.5, 150 mM NaCl, 0.1% Tween 20). The capture surface was prepared in the SFC by standard amine-coupling. An HC30-M chip (Carterra® LSA cat #4279) was activated with a 10-minute injection of freshly prepared 1:1:1 (v/v/v) mixture of 0.4 M EDC+0.1 M NHS+0.1 M MES pH 5.5. A monoclonal mouse anti-HuFc antibody mAb 1.35.1 was prepared at 100 ug/mL in 10 mM sodium acetate pH 4.5 (Carterra, #3628) and coupled for 20 minutes. Excess reactive esters were blocked with a 7-minute injection of 1 M ethanolamine HCl pH 8.5 (Carterra, cat #3626). Final coupling amounts were greater than 1000 response units (RU).

B cell supernatant samples estimated to have an antibody concentration of >100 ng/mL were diluted ~1:6.6 (30 μL supernatant plus 170 μL of Carterra buffer (Hepes buffered Steinberg's Solution (HBS-T); 50 mM HEPES pH 7.5, 150 mM NaCl, 0.1% Tween 20) and printed for 30 minutes using the 96PH. After printing, a non-regenerative kinetic assay was set up by using the SFC to sequentially inject 6 concentrations of target (IL11, Sino Biological/12225-HNCE) at a 1:3 dilution series from 100 nM (100 , 33.33, 11.11, 3.7, 1.23, and 0.41 nM). Dilutions were in running buffer and injected from low to high concentration with no regeneration in between injections. Association was for 10 minutes, with a 20-minute dissociation time.

Immediately after the kinetics cycle, a single 10 minute injection of IL11 receptor (R and D Systems/8895-MR-MTO) at 100 nM in running buffer was performed to measure RL blocking.

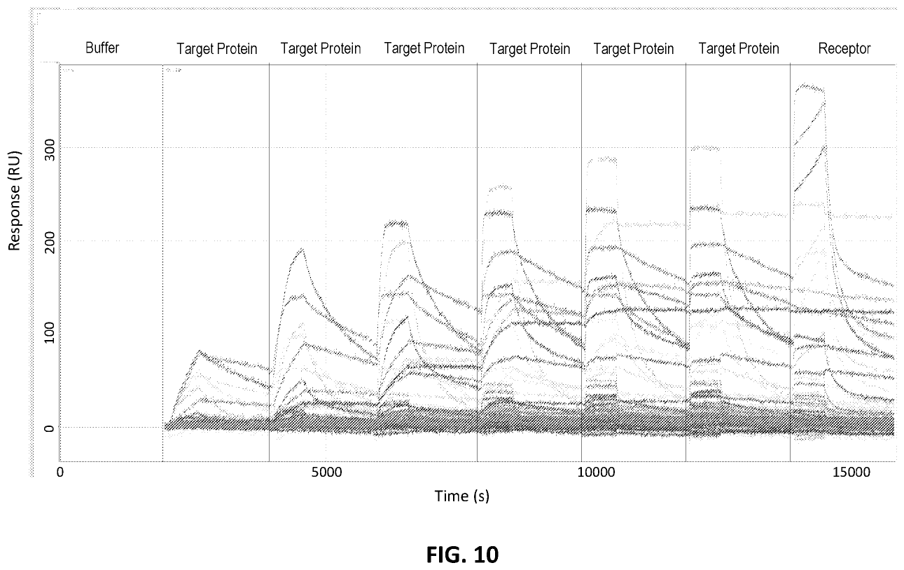

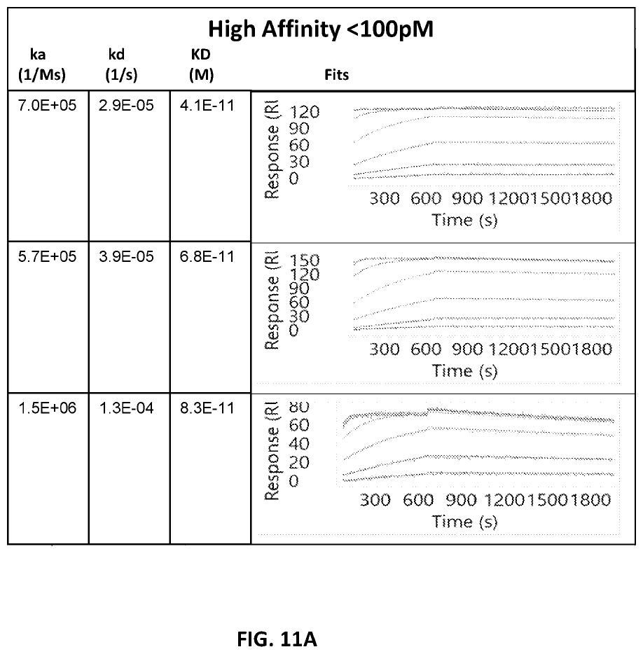

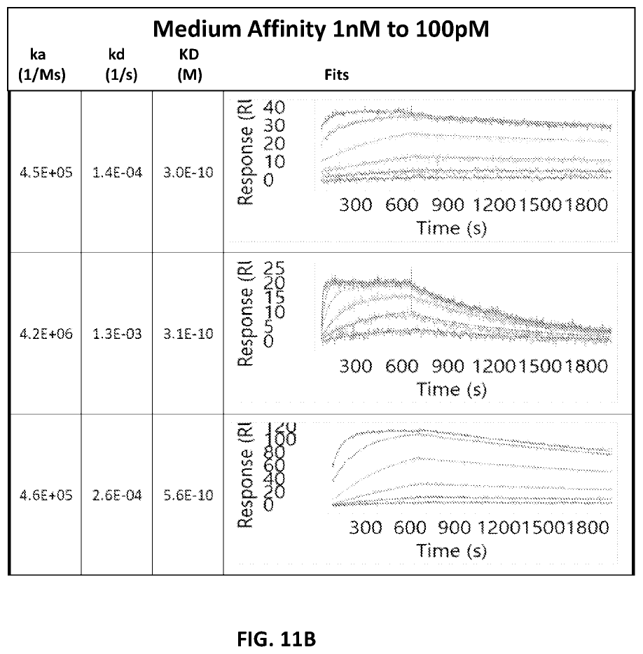

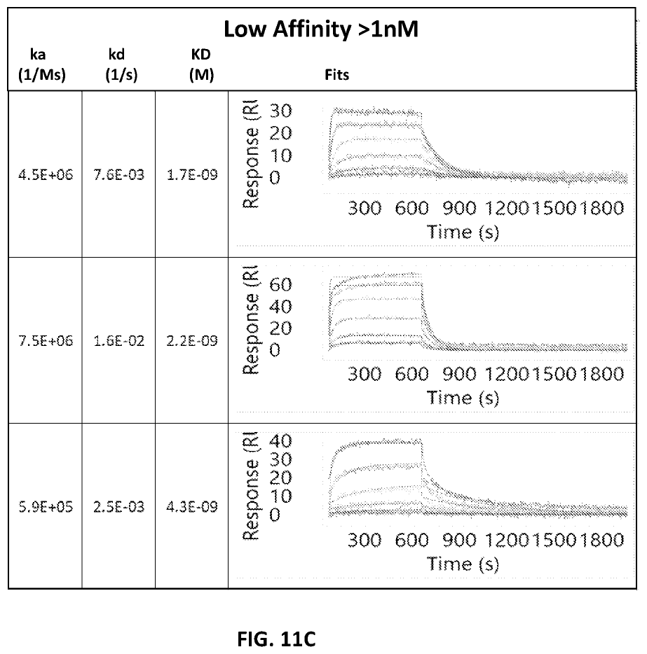

FIG. 10 shows the cumulative sensorgram of this experimental run, where each of binding affinity and RL blocking of the panel of IL-11 mAbs secreted from primary B cells were assessed sequentially in one run over just over 4 hours. FIG. 11 shows affinity sensorgrams for several of the tested antibodies. The far left panel of sensorgrams show high affinity binders (<100 pM), the middle panel shows medium affinity binders (1 nM to 100 pM) and the far right panel shows low affinity binders (>1 nM).

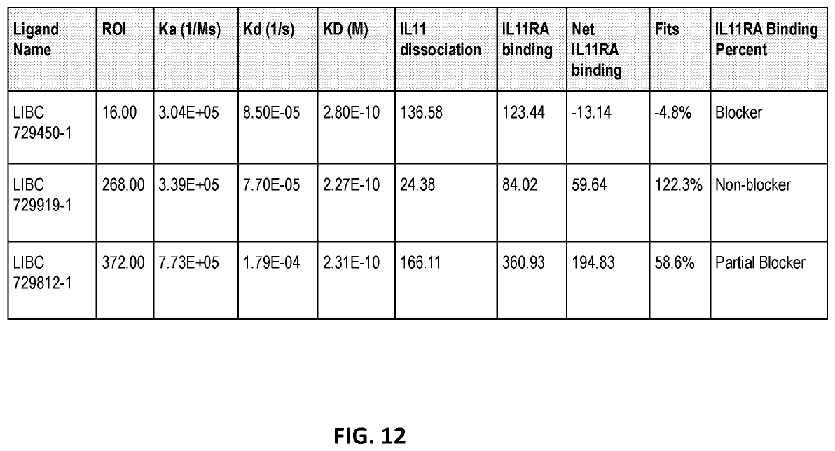

The table of FIG. 12 summarizes the affinity data and blocking activity of three candidate IL-11 antibodies (i.e., antibodies LIBC729450-1, LIBC729919-1, and LIBC729812-1). All three antibodies exhibited high binding affinity, while only LIBC729450-1 functioned as a receptor blocking antibody based on the RU following introduction of target receptor protein (IL-11 Receptor) to the HC30-M chip containing the antibodies bound to their target protein (IL-11). Antibodies LIBC729919-1 and LIBC729812-1 functioned as non-blocker and partial blocker of receptor binding to IL-11 based on their RU values.

In this Example, 96 antibody samples were screened. Of these, 23 antibodies had a desired binding affinity of <40 pM, and of these 23 antibodies, 11 of them blocked IL-11 receptor binding to IL-11. Thus, the method described herein identified 11 potential lead candidates having the desired binding specificity and blocking activity from primary B cell supernatant in just over 4 hours

1. A method of characterizing binding affinity of antibodies from a preparation of B cells, said method comprising:
providing a preparation of non-immortalized B cells, wherein B cells of the preparation secrete immunogen-binding antibodies;
culturing B cells of the preparation individually under conditions effective for said B cells to secrete immunogen-binding antibodies into culture supernatant;
collecting the culture supernatants containing the secreted antibodies from the individually cultured B cells;
exposing each culture supernatant to increasing concentrations of the immunogen, a fragment of the immunogen, or a homolog of the immunogen;
detecting association and dissociation between the secreted antibodies of the culture supernatant and the immunogen, fragment thereof, or homolog thereof at each of the increasing concentrations; and
characterizing binding affinity of said secreted antibodies based on said detecting.

2. The method of claim 1 further comprising:
providing a solid support comprising a plurality of reaction surfaces, wherein each reaction surface comprises a capture reagent immobilized to said surface;
contacting each of the collected culture supernatants with the solid support under conditions effective for secreted antibodies from one collected culture supernatant to bind to the immobilized capture reagent on one reaction surface to form an array of captured antibodies on the solid support, wherein said exposing, detecting, and characterizing are carried out on the solid support.

3. The method of claim 2 further comprising:
contacting the array of capture antibodies with a candidate cross-reactive binding molecule prior to said exposing and
determining presence or absence of an interaction between the candidate cross-reactive binding molecule and the captured antibodies; and
identifying binding cross-reactivity of the captured antibodies as a result of said determining.

4. The method of claim 2 further comprising:
contacting, after said characterizing, the array of capture antibodies with one or more additional binding analytes and
determining presence or absence of an interaction between the one or more additional binding analytes and the captured antibodies to characterize an additional binding characteristic of the captured antibodies.

5. The method of claim 4, wherein the one or more additional binding analytes comprises a binding partner of the immunogen, and blocking activity of the captured antibodies is characterized based on said determining.

6. The method of claim 4, wherein the one or more additional binding analytes is an agent that binds an antibody light chain, and light chain composition of the captured antibodies is characterized based on said determining.

7. The method of claim 2, wherein said detecting and characterizing are carried out using surface plasmon resonance imaging.

8. The method of claim 2, wherein said contacting comprises:
flowing each culture supernatant over a reaction surface of the solid support surface, and
repeating said flowing for at least 15 minutes.

9.-10. (canceled)

11. The method of claim 2, wherein each of the culture supernatants contacted with the reaction surfaces of the solid support comprise a volume of about 150 μl to about 200 μl and contain about 20 ng/mL to about 100 ng/mL of secreted antibody.

12. (canceled)

13. A method of characterizing immunogen-binding antibodies from a preparation of B cells, said method comprising:
providing a preparation of non-immortalized B cells, wherein B cells of the preparation secrete immunogen-binding antibodies;
culturing the B cells of the preparation individually under conditions effective for said B cells to secrete the immunogen-binding antibodies into culture supernatant;
collecting the culture supernatants containing the secreted antibodies from the individually cultured B cells; and
subjecting each collected supernatant to two or more different binding assays to characterize the immunogen-binding antibodies from the preparation of B cells.

14. The method of claim 13, wherein the two or more binding assays are selected from a binding affinity assay, a binding avidity assay, a binding cross-reactivity assay, an assay to determine immunogen binding conditions, a blocking activity assay, an assay to determine antibody chain composition, an epitope binding assay, and an antibody-on-antibody cross competition assay.

15. (canceled)

16. The method of claim 13, wherein said subjecting comprises:
providing a solid support comprising a plurality of reaction surfaces, wherein each reaction surface comprises a capture reagent immobilized to said surface;
contacting each of the collected culture supernatants with the solid support under conditions effective for secreted antibodies from a collected culture supernatant to bind to the immobilized capture reagent on a reaction surface thereby forming an array of captured antibodies on the solid support;
exposing the array of captured antibodies to a first binding analyte;
detecting the presence or absence of an interaction between the first binding analyte and the captured antibodies to determine a first binding characteristic of said antibodies;
repeating, sequentially, said exposing and said detecting with at least a second binding analyte to determine at least a second binding characteristic of said antibodies.

17.-28. (canceled)

29. The method of claim 16 further comprising:
selecting, based on said detecting, one or more candidate antibodies from the array of captured antibodies for sequencing, and
sequencing the selected one or more candidate antibodies.

30. (canceled)

31. The method of claim 16, wherein each collected supernatant contains about 20 ng/mL to about 100 ng/mL of secreted antibody in a volume of about 150 μl to about 200 μl.

32.-36. (canceled)

37. The method of claim 13, wherein said subjecting is carried out in <12 hours.

38. (canceled)

39. The method of claim 1, wherein the preparation of B cells is selected from the group consisting of: a preparation of primary B cells, a preparation of B cells from a subject immunized with the immunogen, and a preparation of B cells from a subject having an autoimmune disorder.

40. The method of claim 13, wherein the preparation of B cells is selected from the group consisting of: a preparation of primary B cells, a preparation of B cells from a subject immunized with the immunogen, and a preparation of B cells from a subject having an autoimmune disorder.

41.-44. (canceled)

45. A method of characterizing binding proteins, said method comprising:
providing one or more samples, each sample comprising about 1 ng to about 25 ng of a binding protein;
contacting the one or more samples with a solid support comprising a plurality of reaction surfaces, wherein each reaction surface comprises a capture reagent immobilized to said surface, and wherein said contacting is carried out under conditions effective for binding proteins from a sample to bind to the immobilized capture reagent on a reaction surface thereby forming an array of captured binding proteins on the solid support;
subjecting the array of captured binding proteins to two or more different binding assays to characterize the binding proteins in each of the one or more samples.

46. The method of claim 45, wherein said subjecting comprises:
exposing the array of captured binding proteins to a first binding analyte;
detecting the presence or absence of an interaction between the first binding analyte and the captured binding proteins to determine a first binding characteristic of said binding proteins; and
repeating, sequentially, said exposing and said detecting with at least a second binding analyte to determine at least a second binding characteristic of said binding protein.

47. The method of claim 45, wherein said contacting comprises:
flowing the sample over a reaction surface of the solid support surface, and
repeating said flowing for at least 15 minutes.

48. The method of claim 47, wherein said repeating is carried out for about 20 minutes to about 40 minutes.

49. (canceled)

50. The method of claim 45, wherein each sample contains about 3 ng to about 20 ng of binding protein.

51. The method of claim 45, wherein each sample contains about 20 ng/mL to about 100 ng/mL of binding protein in a volume of about 150 μl to about 200 μl.

52. The method of claim 45, wherein the two or more binding assays are selected from a binding affinity assay, a binding avidity assay, a binding cross-reactivity assay, an assay to determine immunogen binding conditions, a blocking activity assay, an assay to determine antibody chain composition, an epitope binding assay, and an antibody-on-antibody cross competition assay.

53. (canceled)
